SEQUENCE LISTING (2 sequences)

SEQ ID NO: 1 - protein, 320 aa, Homo sapiens
MAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 2 - protein, 422 aa, Homo sapiens
MVSESHHEALAAPPVTTVATVLPSNATEPASPGEGKEDAFSKLKEKFMNELHKIPLPPWALIAIAIVAVLLVLTCCFCICKKCLFKKKNKKKGKEKGGKNAINMKDVKDLGKTMKDQALKDDDAETGLTDGEEKEEPKEEEKLGKLQYSLDYDFQNNQLLVGIIQAAELPALDMGGTSDPYVKVFLLPDKKKKFETKVHRKTLNPVFNEQFTFKVPYSELGGKTLVMAVYDFDRFSKHDIIGEFKVPMNTVDFGHVTEEWRDLQSAEKEEQEKLGDICFSLRYVPTAGKLTVVILEAKNLKKMDVGGLSDPYVKIHLMQNGKRLKKKKTTIKKNTLNPYYNESFSFEVPFEQIQKVQVVVTVLDYDKIGKNDAIGKVFVGYNSTGAELRHWSDMLANPRRPIAQWHTLQVEEEVDAMLAVKK

(19) United States
(12) Patent Application Publication
(10) Pub. No.: US 2004/0022731 A1
(43) Pub. Date: Feb. 5, 2004

(54) In vivo imaging of apoptosis

(76) Inventors: Alexei Bogdanov, Arlington, MA (US); Eyk Schellenberger, Somerville, MA (US); Alexander Petrovsky, Moscow (RU); Lee Josephson, Reading, MA (US)

Correspondence Address: FISH & RICHARDSON PC, 225 FRANKLIN ST, BOSTON, MA, 02110, US

(21) Appl. No.: 10/424,232

(22) Filed: Apr. 25, 2003

Related U.S. Application Data
(60) Provisional application No. 60/376,052, filed on Apr. 26, 2002.

(51) Int. Cl. A61K 49/00

(52) U.S. Cl. 424/9.6

(57) ABSTRACT

The invention relates to conjugates and methods for in vivo imaging of apoptosis using conjugates of fluorochromes and moieties that bind specifically to apoptotic cells. In specific embodiments, the fluorochrome emits fluorescence in the near-infrared range and is conjugated to a moiety, e.g., a protein such as annexin A5 or synaptotagmin, that binds specifically to apoptotic cells. The methods are non-invasive can be used to obtain images of apoptotic cells in the tissues of living animals, e.g., mammals such as humans.

### CROSS-REFERENCE TO RELATED APPLICATION

This application claims priority under 35 USC §119(e) to U.S. Patent Application Serial No. 60/376,052, filed on Apr. 26, 2002, the entire contents of which are hereby incorporated by reference.

### FIELD OF THE INVENTION

This invention relates to optical imaging, and more particularly to methods and compositions for in vivo imaging of apoptotic cells.

### BACKGROUND OF THE INVENTION

Apoptosis, or programmed cell death, is a fundamentally important process for normal development and in many disease states. Detection of apoptosis in a living organism would allow non-invasive assessment of the extent of cell death in cancer, atherosclerosis, multiple sclerosis and other diseases, as well as the response of these diseases to therapy. However, in vivo apoptosis imaging has proven to be challenging. True apoptosis usually includes fragmentation of genomic DNA, nuclear compaction and fragmentation as well as the drastic compaction of cells (loss of cellular volume). However, early signatures of apoptosis include externalization of aminophospholipids that normally reside at the cytoplasmic side of plasma membrane (cytoplasmic leaflet of membrane bilayer). These pre-apoptotic changes, as a signal of cell stress, may be fully reversible. There is only a limited period of time between the onset of apoptosis and cell removal by macrophages; thus, the presence of apoptotic cells in vivo is usually transient. Also, apoptotic cells often exist in relatively small structures (such as blood vessels) that are smaller than the resolution of radioactive detection methods.

### SUMMARY OF THE INVENTION

The invention is based, in part, on the discovery that certain non-radioactive fluorophore conjugates can be used to detect apoptosis in living animals. Thus, the invention provides non-invasive methods for in vivo apoptosis imaging using non-radioactive conjugates of fluorochromes and moieties that bind specifically to apoptotic cells. In some embodiments, the fluorochrome emits fluorescence in the near-infrared range and is bonded to one or more amino acid residues of a moiety or molecule that binds specifically to apoptotic cells, e.g., a protein such as annexin (e.g., annexin A5), synaptotagmin (e.g., synaptotagmin I), or anti-aminophospholipid antibody (e.g., anti-phosphatidylserine or anti-phosphatidylethanolamine), or an active fragment thereof (e.g., the C2 domain of synaptotagmin, or an antigen-binding fragment of an antibody), that binds specifically to apoptotic cells or to phosphatidylserine-calcium complexes. The conjugates can be used to detect and obtain images of apoptotic cells in the tissues of subjects, such as living humans and animals, e.g., mammals.

In one aspect, the invention features a non-invasive, non-isotopic method for imaging apoptosis in vivo using a fluorescent conjugate. In one embodiment, the method includes administering to a subject a composition including a fluorochrome conjugated to a moiety that binds specifically to apoptotic cells and obtaining a fluorescence image of at least part of the subject (e.g., breast, back, chest, stomach, arm, leg, or other specific organ or specific region of tissue) to detect the site of apoptosis in the subject. In another embodiment, the method includes obtaining a moiety that binds specifically to apoptotic cells; attaching or linking a fluorochrome to the moiety (e.g., via one or more covalent bonds) to form a conjugate; administering the conjugate to a subject; and obtaining a fluorescence image of at least part of the subject to detect the site(s) of apoptosis.

The conjugate includes a moiety (e.g., a protein or active fragment thereof) that binds specifically to apoptotic cells, and a fluorochrome, wherein the fluorochrome is linked, e.g., covalently, to the moiety. In some embodiments, the moiety can be, for example, a protein or an active fragment thereof, e.g., an annexin or an active fragment thereof or synaptotagmin or an active fragment thereof, e.g., in a substantially purified form. In some embodiments, the active fragment is the C2 domain of synaptotagmin. Alternatively, the protein can be an antibody, e.g., an anti-aminophospholipid antibody or an active (e.g., antigen-binding) fragment thereof, such as an anti-phosphatidylserine or anti-phosphatidylethanolamine antibody or antigen-binding fragment thereof, e.g., Fv, Fab or F(ab′)₂.

The fluorochrome can be, for example, a fluorochrome that fluoresces in the near-infrared (NIR) region (in the range of 600-1100 nm), e.g., after excitation in the far-red range of visible light wavelengths. Specific examples include Cy5™, Cy5.5™, Cy7™ or Licor NIR™, ALEXA FLUOR® 680, ALEXA FLUOR® 700, ALEXA FLUOR® 750, IRDye38™, IRDye78™, IRDye80™, indocyanine green, LaJolla Blue™, and Licor NIR™, as well as the fluorochromes disclosed in U.S. Pat. No. 6,083,875, which is incorporated herein by reference in its entirety. The subject can be a human or an animal, for example, a mammal such as a cat, a dog, a mouse, a sheep, a horse, a rat, a rabbit, a pig, or a cow; a bird; a reptile; or a fish.

The conjugate can be administered, for example, orally, parenterally, by inhalation, topically, rectally, nasally, buccally, vaginally, or via an implanted reservoir. The conjugate can also be administered via catheters or through a needle to any tissue.

Fluorescence imaging can be carried out using any suitable imaging camera or device. A number of reflectance and tomographic imaging systems have been developed to detect NIR fluorescence in deep tissues. In some embodiments, the fluorescence image is NIRF imaging, e.g., by fluorescence mediated tomography (FMT) or surface reflectance imaging. In some embodiments, the imaging is carried out using an endoscope.

In another aspect, the invention features conjugates including a moiety that binds specifically to apoptotic cells (e.g., annexin or an active fragment thereof or synaptotagmin or an active fragment thereof, or an anti-aminophospholipid antibody or active fragment thereof), and a fluorochrome covalently bound to the moiety, wherein the moiety and the fluorochrome are present in a stoichiometry of at least 1:2, e.g., “inactive” conjugates useful as controls. In some embodiments, the fluorochrome is a fluorochrome that fluoresces in the near infrared region as described herein.

In another aspect, the invention features conjugates including annexin and a fluorochrome selected from the group consisting of ALEXA FLUOR 680, ALEXA FLUOR 700, ALEXA FLUOR 750, IRDye38, IRDye78, IRDye80, indocyanine green, LaJolla Blue, and Licor NIR. The invention features conjugates comprising synaptotagmin and a fluorochrome selected from those described herein. In other aspects, the invention features conjugates including an anti-aminophospholipid antibody, e.g., an anti-phosphatidylserine antibody or an anti-phosphatidylethanolamine antibody, and a fluorochrome.

As used herein, the terms “fluorochrome” and “fluorochrome dye” both refer to chromophores that are able to absorb energy at a ground state and emit fluorescent light from an excited state. The chromophores can be conjugated with other molecules (e.g., biological macromolecules) to form fluorophore conjugates (e.g., conjugates useful as imaging probes, e.g., NIR fluorescence probes).

The invention provides several advantages. For example, the conjugates have a high affinity for apoptotic cells and are quickly removed from circulation. Conjugation of proteins such as annexin A5 with fluorochromes results in imaging drugs that are similar to the native proteins, in that such conjugation does not significantly alter protein mass, preserves high affinity, and yields a conjugate that can be detected using an excitation source utilizing non-ionizing radiation with the ability to penetrate deep into tissue.

The new methods advantageously allow the use of non-ionizing, NIR radiation (approximately 600-1100 nm) for apoptosis imaging. NIR exhibits tissue penetration of up to tens of centimeters, and can accordingly be used for non-invasively imaging internal tissues (see, e.g., Wyatt, Phil. Trans. R. Soc. London B, 352:701-706, 1997; and Tromberg et al., Phil. Trans. R. Soc. London B, 352:661-667, 1997). Besides being non-invasive, NIR fluorescence imaging methods offer a number of advantages over other imaging methods: they provide generally high sensitivity, do not require exposure of test subjects or lab personnel to ionizing radiation (as can be required by the use of radioactively-labeled proteins), offer the possibility of repeated and frequent use of the imaging procedure, can allow for simultaneous use of multiple, distinguishable conjugates (important in molecular imaging), and offer high temporal and spatial resolution (important in functional imaging and in vivo microscopy, respectively). The conjugates are also very stable; the proteins used in the new methods advantageously do not need to be labeled prior to imaging each time the test is prescribed.

Another advantage is that optical imaging is highly compatible with endoscopic methods of examination and imaging. NIRF methods can be used with, for example, whole body NIRF fluorescence mediated tomography (FMT) imagers (e.g., similar to those described in Ntziachristos et al., Molecular Imaging, 1(2):82-88, 2002; Ntziachristos et al., Nature Medicine, 8:757-760, 2002) or with other NIRF endoscopic methods. Furthermore, annexin A5-based apoptosis imaging can quantitate the extent of apoptosis in target tissues, e.g., tissues defined by the patient's clinical history.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and are not intended to be limiting.

Other features and advantages of the invention will be apparent from the following detailed description, and from the claims.

### BRIEF DESCRIPTION OF DRAWINGS

FIG. 1 is a schematic illustration of a NIRF imaging device, including a 150 W halogen light source, 610-650 nm bandpass filter, a cooled CCD, a 700 nm longpass filter and a computer with imaging software.

FIG. 2 is a schematic illustration of a NIRF imaging device, including a 737 nm/1.5W laser, laser light expander, CCD camera controlled via a camera controller by a computer with imaging software, a mercury lamp, a mercury lamp light expander, and appropriate filters, as well as a light tight black box to house the subject.

FIGS. 3A-3C are a set of representations of a sodium dodecyl sulfate-polyacrylamide gel electrophoresis gel corresponding to purified annexin A5-Cy5.5, with unlabeled annexin A5 in lane 1 (running at about 36 kDa), annexin A5-Cy5.5 in lane 2, and a protein size marker in lane 3. FIG. 3A is a reproduction of a photograph of the gel after BIO-RAD® Silver Staining; FIG. 3B is a near-infrared fluorescence image of the same gel; FIG. 3C is a fusion of FIG. 3A and the negative of FIG. 3B.

FIGS. 4A and 4B are a set of histograms comparing the specificity of annexin A5-FITC (4A) and annexin A5-Cy5.5 (4B) for apoptotic cells by FACS analysis; the gray areas of the histograms correspond to untreated control Jurkat T cells, and the white areas correspond to camptothecin-treated cells.

FIGS. 5A and 5B are bar charts comparing the fluorescence heights of non-apoptotic and apoptotic T cells.

FIGS. 6A and 6B are a set of dot plots of double labeled with annexin A5-FITC and annexin A5-Cy5.5; FIG. 6A shows untreated control cells with a high fraction of annexin A5-FITC and annexin A5-Cy5.5 negative, non-apoptotic cells (lower left quadrant 92.0%, upper right 7.4%), whereas the camptothecin treated cells in FIG. 6B have a high fraction of annexin A5-FITC and annexin A5-Cy5.5 positive, apoptotic cells (lower left quadrant 36.0%, upper right 58.9%).

FIGS. 7A and 7B are a histogram (7A) and a bar chart (7B) corresponding to a competition assay with annexin A5-Cy5.5 with different ratios of Cy5.5 dye per protein molecule.

FIG. 8 is a graph showing the time-dependence of NIRF signal intensity measured in treated and non-treated animals bearing 9L-GFP tumors.

FIGS. 9A and 9B are reproductions of photographs of bilateral Lewis lung carcinoma tumors after CPA treatment (arrows), 2 hours after injection of annexin A5-Cy5.5. 9A: visible light; 9B: NIRF images.

FIG. 10 is a reproduction of a set of eight photographs of gliosarcoma tumors depicting two animals from independent experiments (the right and left columns). Row A: green fluorescent protein signal; Row B: NIRF image prior to injection of the active annexin 5A; Row C: NIRF image after injection of the active annexin 5A, but before cyclophosphamide treatment; Row D: NIRF image after cyclophosphamide treatment.

FIGS. 11A-F are reproductions of photographs of chemoresistant CR-LLC and chemosensitive Lewis lung carcinoma tumors. 11A: Visible light image after injection of active annexin 5A; 11B: raw NIRF image of 11A; 11C: map of NIRF intensity superimposed on the white-light image; 11D: raw image of carcinomas after injection of inactive annexin 5A; 11E: TUNEL analysis of the LLC tumor; 11F: TUNEL analysis of CR-LLC tumor.

FIG. 12 is a bar graph showing the effect of treatment with CPA on chemosensitive (LLC) and chemoresistant (CR-LLC) Lewis lung carcinoma tumors, as measured by NIRF.

FIG. 13 is a bar graph showing the time-dependence of tumor NIRF signal intensity with the chemosensitive Ds Red2 Lewis lung carcinoma model. Open bars: cyclophosphamide (CPA)-treated/inactive Cy-annexin (n=3). Dotted bars: no CPA/active Cy-annexin V (n=6). Black bars: CPA treated/active Cy-annexin (n=6).

### DETAILED DESCRIPTION OF THE INVENTION

The invention is directed to methods of use of fluorescent conjugates for in vivo imaging of apoptosis. The conjugates include moieties, e.g., proteins or protein fragments (e.g., annexin A5 or synaptotagmin or active fragments thereof) or other molecules, that bind specifically to apoptotic cells, conjugated with fluorochromes (e.g., NIR fluorochromes such as Cy5™, Cy5.5™, Cy7™ or Licor NIR™, Alexa Fluor® 680, Alexa Fluor® 700, Alexa Fluor® 750, IRDye38™, IRDye78™, IRDye80™, indocyanine green, LaJolla Blue™, and Licor NIR™, and the fluorochromes disclosed in U.S. Pat. No. 6,083,875). Moieties that bind specifically to apoptotic cells have a high affinity for apoptotic cells and, in a mixed population of apoptotic and viable cells, bind preferentially to apoptotic cells and do not substantially bind to on-apoptotic, viable cells.

The methods can include, for example, administering the conjugates to an animal and then detecting photons emitted in the peripheral and deep tissues (e.g., from depths of microns to centimeters from the surface, e.g., at least 1 cm, 2 cm, 3 cm, 4 cm, 5 cm, 6 cm, 7 cm, 8 cm, 9 cm, 10 cm, 12 cm, 15 cm) of the animal after excitation at the proper excitation wavelength for the particular fluorochrome.

Without intending to be bound by any particular mechanism or theory of action, it is believed that the externalization of aminophospholipids that normally reside on the cytoplasmic side of cellular plasma membranes is among the earliest signatures of apoptosis, and that the conjugates recognize cells undergoing apoptosis by recognizing a high number of phosphatidylserine molecules on their surface. The expression of aminophospholipids on the cell surface allows efficient detection of apoptosis. Annexin A5, for example, binds to phosphatidylserine-calcium complexes found on the surface of cells during early apoptosis.

Preparation of the Conjugates

The conjugates can be prepared by combining an optical imaging fluorescent dye or fluorochrome with a moiety (e.g., a protein or other molecule) that selectively and/or specifically binds to apoptotic cells, in the presence of a coupling agent, or by using an activated analog of the dye or fluorochrome, and then allowing the dye or fluorochrome to react to form a bond or link (e.g., a covalent bond or an electrostatic interaction such as an ionic or hydrophobic interaction) between the protein or other molecule and the dye or fluorochrome. The bond can also be formed between the dye or fluorochrome and groups present in proteins, such as sugars, phospholipids, fatty acids, and/or other prosthetic groups, as a result of post-translational modification. For example, N-hydroxsuccinimide esters or isothiocyanates of dyes can be reacted with protein amino groups, and maleimide groups of dyes are reacted with protein sulfhydryl groups.

A linker molecule can also be used to attach one or more fluorochromes to the binding moiety. Suitable linkers include aminoacaproic acid, aminohexanoic acids, heterobifunctional polyethyleneglycols bearing a terminal amino-function, polyethylene glycol vinyl sulfonates, branched polyethylene glycols, and aminated dextrans, inter alia. The preferred molecular mass range of the linker is 200-20,000 D. The resultant conjugates can then be purified to separate free, non-bound dye or fluorochrome from the conjugates.

Experiments with over-modified annexin 5A indicate that the presence of multiple large indocyanine residues negatively affect the binding of annexin 5A to phosphatidylserine. Several approaches can be used to deal with this issue. For example, the binding site can be reversibly protected, e.g., with diacylphosphatidylserine or phosphatidylethanolamine liposomes. In addition, fusion proteins of annexin and a carrier protein (for example, serum albumin) can be prepared. For example, a cDNA of annexin or a fragment of cDNA encoding a binding center amino acid sequence can be ligated with a cDNA of a carrier protein in such a manner that the cDNA of annexin encodes the N-terminal portion of the fusion protein and the carrier protein encodes the C-terminal portion of said fusion protein. The protein can be expressed, e.g., in a suitable bacterial host or in insect or mammalian cells and purified using standard biochemical procedures (e.g. His-tag approach). The purified protein could be then combined with lipids that reversibly block the binding site on the annexin. Fluorescent dye activated analogs can then be added to modify exposed amino groups or SH-groups of cysteine. Alternatively, a linker group can be used to keep the fluorophores further away from the binding site of the moiety.

Table 1 provides examples of near-infrared fluorochromes that are commercially available. In addition, several other near-infrared fluorochromes have been described that are not presently commercially available, see U.S. Pat. No. 6,083,486 to Weissleder et al.; Zabeer et al., Molecular Imaging, 1:354-364, 2002; Becker et al., Nature Biotechnol., 19:327-331, 2001; Licha et al., Bioconj Chem, 12:44-50, 2001. Quantum dots that fluoresce in the near-infrared range could also be used (see, e.g., Watson et al., BioTechniques, 34(2):296-300, 302-3, 2003; Goldman et al., J. Am. Chem. Soc. 124(22):6378-82, 2002; Han et al., Nat. Biotechnol., 19(7):631-5, 2001; Chan et al., Science, 281(5385):2016-8, 1998). At the present time, only one NIRF compound, indocyanine green (ICG), is approved for use in clinical trials or practice. One of skill in the art would appreciate that a large number of fluorochromes with different chemical and optical properties can be used in the new methods.

TABLE 1
NIRF Fluorochromes

| | Ex & Em | Extinct. | |
|---|---|---|---|
| Fluorochrome | Max (nm) | Coeff. (x 10⁻³) | Source |
| Cy5 ™ | 649/670 | 250 | Amersham |
| Cy5.5 ™ | 675/694 | 250 | Amersham |
| Cy7 ™ | 743/767 | 200 | Amersham |
| Alexa Fluor ® 680 | 679/702 | 184 | Molecular |
| | | | Probes |
| Alexa Fluor ® 700 | 696/719 | 192 | Molecular |
| | | | Probes |
| Alexa Fluor ® 750 | 752/779 | 240 | Molecular |
| | | | Probes |
| IRDye ™ 38 | 778/806 | 179 | LiCor |
| IRDye ™ 78 | 768/796 | 220 | LiCor |
| IRDye ™ 80 | 767/791 | 250 | LiCor |
| LaJolla Blue ™ | 680/700 | 170 | Diatron |
| ICG | 780/812 | 115 | Akorn and |
| | | | others |

The moiety that specifically binds to apoptotic cells can be an annexin or an active variant or fragment thereof. As used herein, the term “annexin” refers to a member of a family of structurally related proteins whose common property is calcium-dependent binding to phospholipids. Members of the family include the A annexins (e.g., annexins A1-A13), B annexins (e.g., annexin B12), C annexins (e.g., annexin C1), D annexins (e.g., annexin D), and E annexins (e.g., annexin E1-E3). In one embodiment, the annexin is annexin A5 (also referred to herein as “annexin V”; genbank accession no. NM_{—}001154; SEQ ID NO:1) or an active variant thereof. Active fragments of annexins can also be used, e.g., fragments that retain the ability to bind phospholipids.

The moiety that specifically binds to apoptotic cells can also be a synaptotagmin or an active variant or fragment thereof. As used herein, the term “synaptotagmin” refers to a member of a family of integral membrane proteins that contain C2 domains that bind phospholipids. Members of the family include Syt1-13. In humans, the family includes Syt1-7 and 12-13. In one embodiment, the synaptotagmin is synaptotagmin I, or SYT1 (genbank accession no. M55047; SEQ ID NO:2), or an active variant thereof. Active fragments of synaptotagmin can also be used, e.g., fragments that retain the ability to bind phospholipids, e.g., the C2 domain or variants thereof. Other C2 domains that specifically bind apoptotic cells can also be used within the scope of the invention, including those C2 domains listed at internet address us.expasy.org/cgi-bin/prosite-search-ac?PS50004.

As used herein, the term “active variant” refers to a polypeptide that is a variant of a selected protein (e.g., a native or wildtype protein) that retains a relevant biological activity, e.g., binding ability. A variant can differ from the selected protein at one or more residues, e.g., can have one or more conservative amino acid substitutions. A variant can be a naturally occurring polypeptide, e.g., a polypeptide that occurs in nature (e.g., a natural protein), or a genetically modified variant. As one example, an active variant of annexin can be at least 60%, 70%, 80%, 90%, 95%, or 99% identical to the sequence of an annexin, e.g., SEQ ID NO:1 that retains the ability to bind phospholipids. The active variants of annexin should contain at least one conserved annexin-calcium/phospholipid binding repeat (e.g., lipocortin domain). As one example, an active variant of annexin can be a genetically modified annexin 5A, e.g., as disclosed in Tait et al., Bioconjug Chem, 11, 918-925, 2000. As another example, an active variant of synaptotagmin can be at least 60%, 70%, 80%, 90%, 95%, or 99% identical to the sequence of a synaptotagmin, e.g., SEQ ID NO:2 that retains the ability to bind phospholipids. Variants can include homologs, e.g., homologs of annexin or synaptotagmin that retain the ability to bind phospholipids. Calculations of homology or sequence identity between sequences (the terms are used interchangeably herein) can be performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, 40%, 50%, 60%, 70%, 80%, 90%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm, e.g., the Needleman and Wunsch ((1970) J. Mol. Biol. 48:444-453) algorithm that has been incorporated into the GAP program in the GCG software package (available on the world wide web at gcg.com), using either a Blossum 62 matrix or a PAM250 matrix, and a gap weight of 16, 14, 12, 10, 8, 6, or 4 and a length weight of 1, 2, 3, 4, 5, or 6. Other methods of comparison are known in the art.

The annexin and synaptotagmin nucleic acid and protein sequences can be used as a “query sequence” to perform a search against public databases to, for example, identify variants. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to reference nucleic acid molecules. BLAST protein searches can be performed with the XBLAST program, score=50, wordlength=3 to obtain amino acid sequences homologous to the reference protein. To obtain gapped alignments for comparison purposes, Gapped BLAST can be utilized as described in Altschul et al., Nucleic Acids Res., 25:3389-3402, 1997. When utilizing BLAST and Gapped BLAST programs, the default parameters of the respective programs (e.g., XBLAST and NBLAST) can be used. See, for example, world wide web address ncbi.nlm.nih.gov.

As used herein, a “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

As used herein, the term “active fragment” refers to a polypeptide that is a portion of a larger protein that retains a relevant biological activity, e.g., binding ability. For example, an active fragment of synaptotagmin is the C2 domain, that retains the ability to bind to phosphatidylserine. The active fragment can also be a variant, e.g., can differ from the selected protein at one or more residues, e.g., can have one or more conservative amino acid substitutions.

The moiety that specifically binds to apoptotic cells can also be an anti-aminophospholipid antibody, e.g., an anti-phosphatidylserine or anti-phosphatidylethanolamine antibody, or an active variant or fragment thereof. Such antibodies can be obtained, e.g., by methods known in the art, e.g., the methods described herein. In addition, a number of such antibodies are commercially available, e.g., from Corgenix, Inc. (Denver, Colo.) or Midwest Hemostasis and Thrombosis Laboratories, Inc. (Muncie, Ind.). An active fragment of an antibody can be an Fv, Fab or F(ab′)₂ that retains the ability to bind antigen, e.g., phosphatidylserine or phosphatidyl-ethanolamine. Such fragments can be produced from the antibody using techniques well established in the art (see, e.g., Rousseaux et al., Methods Enzymol., 121:663-69, 1986). For example, the F(ab′)2 fragments can be produced by pepsin digestion of the antibody molecule, and the Fab fragments can be generated by reducing the disulphide bridges of the F(ab′)2 fragments. A number of methods are known in the art for humanizing or de-immunizing antibodies to reduce the risk of anti-antibody reactions such as human anti-mouse antibody (HAMA) reactions. Active variants of anti-aminophospholipids can include, for example, humanized or de-immunized versions of non-human antibodies, e.g., mouse or rabbit, as described herein.

Methods of Making Antibodies

Antibodies are immunoglobulin molecules and immunologically active (e.g., antigen-binding) portions of immunoglobulin molecules. Examples of fragments of immunoglobulin molecules include fragments of an antibody, e.g., Fv, F(ab) or F(ab′)₂ portions, which can specifically bind to apoptotic cells, e.g., to aminophospholipid markers of apoptosis such as phosphatidylserine or phosphatidylethanolamine. Fragments can be generated by treating an antibody with an enzyme such as pepsin. The term monoclonal antibody or monoclonal antibody composition refers to a population of antibody molecules that contain only one species of an antigen binding site capable of immunoreacting with a particular epitope of a polypeptide or protein. A monoclonal antibody composition thus typically displays a single binding affinity for the protein to which it specifically binds.

Immunization

Polyclonal and monoclonal antibodies against apoptotic cells can be raised by immunizing a suitable subject (e.g., a rabbit, goat, mouse or other mammal) with an immunogenic preparation that contains a suitable immunogen. Immunogens include phosphatidylserine or phosphatidylethanolamine, or artificial protein antigens (e.g., hemocyanin) covalently modified with phosphoryl serine groups or oxidized unsaturated or polyunsaturated diacyl phosphatidyl serine. Typically, the immunogen is phosphatidylserine.

The antibodies raised in the subject can then be screened to determine if the antibodies bind to apoptotic cells. Such antibodies can be further screened in the assays described herein. For example, these antibodies can be assayed to determine if they demonstrate binding patterns similar to an annexin, e.g., annexin 5A, or synaptotagmin. Suitable methods to identify an antibody with the desired characteristics are described herein and are known in the art.

The unit dose of immunogen (e.g., phosphatidylserine or phosphatidylethanolamine) and the immunization regimen will depend upon the subject to be immunized, its immune status, and the body weight of the subject. To enhance an immune response in the subject, an immunogen can be administered with an adjuvant, such as Freund's complete or incomplete adjuvant. Immunization of a subject with an immunogen as described above induces a polyclonal antibody response. The antibody titer in the immunized subject can be monitored over time by standard techniques such as an ELISA using an immobilized antigen, e.g., phosphatidylserine or phosphatidylethanolamine.

Other methods of raising antibodies against apoptotic cells include using transgenic mice that express human immunoglobulin genes (see, e.g., Wood et al. PCT publication WO 91/00906, Kucherlapati et al. PCT publication WO 91/10741; or Lonberg et al. PCT publication WO 92/03918). Alternatively, human monoclonal antibodies can be produced by introducing an antigen into immune deficient mice that have been engrafted with human antibody-producing cells or tissues (e.g., human bone marrow cells, peripheral blood lymphocytes (PBL), human fetal lymph node tissue, or hematopoietic stem cells). Such methods include raising antibodies in SCID-hu mice (see Duchosal et al. PCT publication WO 93/05796; U.S. Pat. No. 5,411,749; or McCune et al. Science 241:1632-1639, 1988)) or Rag-1/Rag-2 deficient mice. Human antibody-immune deficient mice are also commercially available. For example, Rag-2 deficient mice are available from Taconic Farms (Germantown, N.Y.).

Hybridomas

Monoclonal antibodies can be generated by immunizing a subject with an immunogen. At the appropriate time after immunization, e.g., when the antibody titers are at a sufficiently high level, antibody producing cells can be harvested from an immunized animal and used to prepare monoclonal antibodies using standard techniques. For example, the antibody producing cells can be fused by standard somatic cell fusion procedures with immortalizing cells such as myeloma cells to yield hybridoma cells. Such techniques are well known in the art, and include, for example, the hybridoma technique as originally developed by Kohler and Milstein, Nature, 256:495-497, 1975), the human B cell hybridoma technique (Kozbar et al., Immunology Today, 4:72, 1983), and the EBV-hybridoma technique to produce human monoclonal antibodies (Cole et al., Monoclonal Antibodies and Cancer Therapy, Alan R. Liss, Inc. pp. 77-96 (1985)). The technology for producing monoclonal antibody hybridomas is well known.

Monoclonal antibodies can also be made by harvesting antibody-producing cells, e.g., splenocytes, from transgenic mice expressing human immunoglobulin genes and that have been immunized with an appropriate antigen. The splenocytes can be immortalized through fusion with human myelomas or through transformation with Epstein-Barr virus (EBV). These hybridomas can be made using human B cell-or EBV-hybridoma techniques described in the art (see, e.g., Boyle et al., European Patent Publication No. 0 614 984).

Hybridoma cells producing a monoclonal antibody that specifically binds to apoptotic cells are detected by screening the hybridoma culture supernatants by, for example, screening to select antibodies that specifically bind to apoptotic cells, or to a marker of apoptosis, e.g., an aminophospholipid such as phosphatidylserine or phosphatidylethanolamine.

Hybridoma cells that produce monoclonal antibodies that test positive in the screening assays described herein can be cultured in a nutrient medium under conditions and for a time sufficient to allow the hybridoma cells to secrete the monoclonal antibodies into the culture medium, to thereby produce whole antibodies. Tissue culture techniques and culture media suitable for hybridoma cells are generally described in the art (see, e.g., R. H. Kenneth, in Monoclonal Antibodies: A New Dimension In Biological Analyses, Plenum Publishing Corp., New York, N.Y. (1980). Conditioned hybridoma culture supernatant containing the antibody can then be collected.

Recombinant Combinatorial Antibody Libraries

Monoclonal antibodies can be engineered by constructing a recombinant combinatorial immunoglobulin library and screening the library with an appropriate antigen, e.g., phosphatidylserine or phosphatidylethanolamine. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Briefly, the antibody library is screened to identify and isolate phages that express an antibody that specifically binds to the desired antigen. In one embodiment, the primary screening of the library involves screening with immobilized phosphatidylserine or phosphatidylethanolamine.

Following screening, the display phage is isolated and the nucleic acid encoding the selected antibody can be recovered from the display phage (e.g., from the phage genome) and subcloned into other expression vectors by well known recombinant DNA techniques. The nucleic acid can be further manipulated (e.g., linked to nucleic acid encoding additional immunoglobulin domains, such as additional constant regions)and/or expressed in a host cell.

Chimeric and Humanized Antibodies

Recombinant forms of antibodies, such as chimeric and humanized antibodies, can also be prepared to minimize the response by a human patient to the antibody. When antibodies produced in non-human subjects or derived from expression of non-human antibody genes are used therapeutically in humans, they are recognized to varying degrees as foreign, and an immune response may be generated in the patient. One approach to minimize or eliminate this immune reaction is to produce chimeric antibody derivatives, i.e., antibody molecules that combine a non-human animal variable region and a human constant region. Such antibodies retain the epitope binding specificity of the original monoclonal antibody, but may be less immunogenic when administered to humans, and therefore more likely to be tolerated by the patient.

Chimeric monoclonal antibodies can be produced by recombinant DNA techniques known in the art. For example, a gene encoding the constant region of a non-human antibody molecule is substituted with a gene encoding a human constant region (see Robinson et al., PCT Patent Publication PCT/US86/02269; Akira, et al., European Patent Application 184,187; or Taniguchi, M., European Patent Application 171,496).

A chimeric antibody can be further “humanized” by replacing portions of the variable region not involved in antigen binding with equivalent portions from human variable regions. General reviews of “humanized” chimeric antibodies are provided by Morrison, S. L. Science, 229:1202-1207, 1985 and by Oi et al. BioTechniques, 4:214, 1986. Such methods include isolating, manipulating, and expressing the nucleic acid sequences that encode all or part of an immunoglobulin variable region from at least one of a heavy or light chain. The cDNA encoding the humanized chimeric antibody, or fragment thereof, can then be cloned into an appropriate expression vector. Suitable “humanized” antibodies can be alternatively produced by (complementarity determining region (CDR) substitution (see U.S. Pat. No. 5,225,539; Jones et al., Nature, 321:552-525, 1986; Verhoeyan et al., Science, 239:1534, 1988; and Beidler et al, J. Immunol., 141:4053-4060, 1988).

Epitope imprinting can also be used to produce a “human” antibody polypeptide dimer that retains the binding specificity of antibodies specific for apoptotic cells. Briefly, a gene encoding a non-human variable region (VH) with specific binding to an antigen and a human constant region (CHI), is expressed in E. coli and infected with a phage library of human VλCλ genes. Phage displaying antibody fragments are then screened for binding to the 40 kDa protein. Selected human Vλ genes are re-cloned for expression of VλCλ chains and E. coli harboring these chains are infected with a phage library of human VHCH1 genes and the library is subject to rounds of screening with antigen coated tubes. See Hoogenboom et al., PCT publication WO 93/06213.

Administration of the Conjugates

Pharmaceutically acceptable carriers and vehicles can be used to form a composition or pharmaceutical formulation including the conjugates described herein.

Useful carriers and vehicles include, but are not limited to, ion exchangers, alumina, aluminum stearate, lecithin, serum proteins such as albumin, buffer substances such as phosphate, glycine, sorbic acid, potassium sorbate, tris(hydroxymethyl)amino methane (“TRIS”), partial glyceride mixtures of fatty acids, water, salts or electrolytes, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polypropylene block co-polymers, sugars such as glucose, and suitable cryoprotectants.

The pharmaceutical compositions of the conjugates described herein can be in the form of a sterile injectable preparation. The possible vehicles or solvents that can be used to make injectable preparations include water, Ringer's solution, and isotonic sodium chloride solution, and 5% D-glucose solution (D5W). In addition, oils such as mono- or di-glycerides and fatty acids such as oleic acid and its derivatives can be used.

The conjugates and pharmaceutical compositions of the present invention can be administered orally, parenterally, by inhalation, topically, rectally, nasally, buccally, vaginally, or via an implanted reservoir. The term “parenteral administration” includes intravenous, intramuscular, intra-articular, intrasynovial, intrasternal, intrathecal, intraperitoneal, intracisternal, intrahepatic, intralesional, and intracranial injection or infusion techniques. The conjugates can also be administered via catheters or through a needle to any tissue.

For ophthalmic use, the pharmaceutical compositions of the invention can be formulated as micronized suspensions in isotonic, pH-adjusted, sterile saline. Alternatively, the compositions can be formulated in ointments such as petrolatum.

For topical application, the new pharmaceutical compositions can be formulated in a suitable ointment, such as petrolatum. Topical application for the lower intestinal tract or vagina can be achieved by a suppository formulation or enema formulation.

The formulation of the conjugate can also include an antioxidant or some other chemical compound that prevents or reduces the degradation of the baseline fluorescence, or preserves the fluorescence properties, including, but not limited to, quantum yield, fluorescence lifetime, and excitation and emission wavelengths. These antioxidants or other chemical compounds can include, but are not limited to, melatonin, dithiothreitol (dTT), deferoxamine (DFX), methionine, and N-acetyl cysteine.

Dosing of the invention will depend on a number of factors including the instruments' sensitivity, as well as a number of subject-related variables, including animal species, age, body weight, mode of administration, sex, diet, time of administration, and rate of excretion.

Prior to use of the invention or any pharmaceutical composition of the conjugates, the subject can be treated with an agent or regimen to enhance the imaging process. For example, a subject can be put on a special diet prior to imaging to reduce any auto-fluorescence or interference from ingested food, such as a low pheophorbide diet to reduce interference from fluorescent pheophorbides that are derived from some foods, such as green vegetables. Alternatively, a cleansing regimen can be used prior to imaging, such as those cleansing regimens that are used prior to colonoscopies and include use of agents such as Visicol™.

The subject can also be treated with pharmacological modifiers to improve image quality. For example, using low dose enzymatic inhibitors (secondary to proportionally lowering enzymatic activity of already low-enzymatic activity normal tissues to a greater extent than enzymatically-active pathological tissues) can improve the target-to-background ratio during disease screening. As another non-limiting example, pretreatment with methotrexate to relatively increase uptake in abnormal tissue (i.e., metabolically active cancers) in conjunction with folate-based targeted delivery can be employed.

Uses of In Vivo Imaging

The in vivo imaging methods described herein can be used, for example, to detect apoptosis in a subject, and to evaluate the effect of administering a treatment, both in individual patients and in clinical trials. The methods can be used, e.g., to monitor rates of apoptosis over time, and to detect absolute levels of damage. For example, the methods can be used to detect early signs of apoptosis, e.g., apoptosis in tumors, to evaluate the effect of, for example, cancer treatments such as chemotherapeutic agents, radiation treatments, hormonal or anti-hormonal agents, and anti-angiogenic therapies, and thus to guide clinical care. The sensitivity of the methods allows earlier and rapid detection of apoptosis, enhancing the likelihood of finding an effective therapy.

The methods can also be used to monitor autoimmune conditions such as rheumatoid arthritis and system lupus erythematosus, which are characterized by disturbances in the apoptotic process, primarily in lymphocytic cells.

Progress and treatment of conditions associated with acute apoptosis and/or necrosis, e.g., hypoxic-ischemic injuries such as stroke or myocardial infarction can also be monitored using the methods described herein, and used, e.g., to guide therapeutic choices, e.g., to evaluate the effectiveness of administering anti-apoptotic agents such as caspase inhibitors. Acute organ rejection after transplantation can also be monitored using the instant methods, and the effects of administering immunosuppressive drugs or other agents can be monitored. Cellular damage associated with bacterial and/or viral infections can also be assessed, treatments chosen (e.g., which agent or other treatment to administer), and the efficacy of treatments evaluated. The progress of neurodegenerative diseases characterized by chronic apoptosis including amyotrophic lateral sclerosis, motor-neuronal degeneration, multiple sclerosis, and Alzheimer's, Parkinson's, and Huntington's disease can also be monitored using the methods. For example, the extent and localization of damage can be determined, as well as the progress of the disease over time, and the choice and effect of therapeutic agents can be determined.

NIR Fluorescence Imaging

In NIR fluorescence (“NIRF”) imaging, the source of excitation light is generally a filtered light source or a laser with a defined bandwidth. The excitation light travels through body tissues. When it encounters an NIR fluorescent molecule (i.e., a “contrast agent”), the excitation light is absorbed. The fluorescent molecule then emits light that has, for example, detectably different spectral properties (e.g., a slightly longer wavelength) from the excitation light. NIR technology offers unique advantages for imaging pathology, because tissues and blood have a high transmittance in the near-infrared range (700-850 nm) as opposed to visible light, and neither water nor many naturally occurring fluorochromes absorbs significantly in this region. Thus, NIR light penetrates tissues more efficiently than visible light or photons in the infrared region. In addition, there is lower interference of scattered excitation with far-red light. As a result, the fluorescence signal excited in the deeper layers of tissue can be acquired (reviewed in Hawrysz and Sevick-Muraca, Neoplasia, 2:388-417, 2000). In general, images of tissues can be obtained at a depth of up to tens of centimeters, e.g., at least 1 cm, 2 cm, 3 cm, 4 cm, 5 cm, 6 cm, 7 cm, 8 cm, 9 cm, 10 cm, 12 cm, 15 cm, 18 cm, or 20 cm.

In Vivo Near-Infrared Imaging

Although the invention involves novel methods, general principles of fluorescence, optical image acquisition, and image processing can be applied in the practice of the invention. For a review of optical imaging techniques, see, e.g., Alfano et al., Ann. NY Acad. Sci., 820:248-270, 1997.

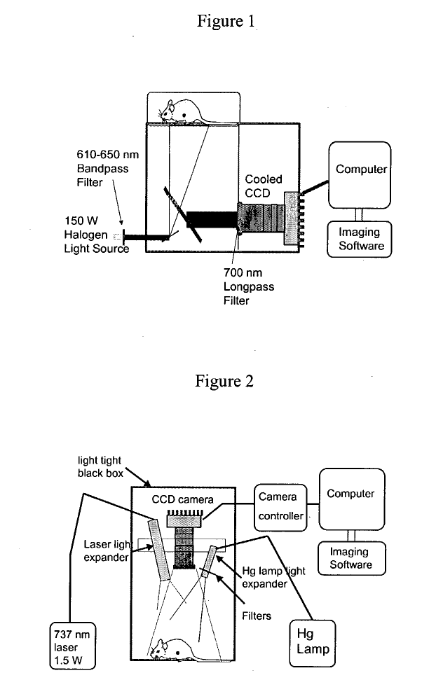

An imaging system useful in the practice of this invention typically includes three basic components: (1) a source of near-infrared or other light of a wavelength suitable to cause the fluorophore to fluoresce, (2) an apparatus for separating or distinguishing emissions from light used for fluorophore excitation, and (3) a detection system. See, e.g., Weissleder et al., Nat. Biotechnol., 17:375-8, 1999. For example, an imaging system such as is shown in FIG. 1 can be assembled using a Kodak ImageStation 440 imaging station and an external low-power excitation source. This surface reflectance fluorescent (SRF) imaging device was used to generate the in vivo data described herein. The device includes a white light halogen source producing a low intensity light (1 μW/cm² at 650 nm). In surface reflectance imaging, light at a wavelength needed to excite the fluorochrome is applied to the surface of the animal positioned on the glass platen, and an image is made of the Stokes-shifted (“fluorescent”) light. Bandpass filters can be used for wavelength discrimination. More sophisticated systems, such as the one shown in FIG. 2, which features high power laser excitation from the laser (1 mW/cm² at 737 nm) and a mercury lamp for light at other wavelengths, more varied filters and an improved CCD camera, can also be used.

Typically, the light source provides monochromatic (or substantially monochromatic) near-infrared light when using NIR fluorophores. The light source can be a suitably filtered white light, e.g., bandpass-filtered light from a broadband source. For example, light from a 150-watt halogen lamp can be passed through a suitable bandpass filter commercially available from Omega Optical (Brattleboro, Vt.). In some embodiments, the light source is a laser. See, e.g., Boas et al., Proc. Natl. Acad. Sci. USA, 91:4887-4891, 1994; Ntziachristos et al., Proc. Natl. Acad. Sci. USA, 97:2767-2772, 2000; Alexander, J. Clin. Laser Med. Surg., 9:416-418, 1991. Information on near-infrared lasers for imaging can also be found on the Internet (e.g., at imds.com) and various other known sources.

A high pass or bandpass filter (700 nm) can be used to separate optical emissions from excitation light. A suitable high pass or bandpass filter is commercially available from Omega Optical. Where the fluorochrome consists of one or more quantum dots, a single excitation wavelength can be used to excite multiple different fluorochromes on a single probe or multiple probes (with different activation sites), and spectral separation with a series of bandpass filters, diffraction grating, or other means can be used to independently read the different activations.

In general, the light detection system can include light-gathering/image-forming and light-detection/image-recording components. Although the light-detection system can be a single integrated device that incorporates both components, the light-gathering/image-forming and light-detection/image-recording components will be discussed separately. However, a recording device may simply record a single (time varying) scalar intensity instead of an image. For example, a catheter-based recording device can record information from multiple sites simultaneously (i.e., an image), or can report a scalar signal intensity that is correlated with location by other means (such as a radio-opaque marker at the catheter tip, viewed by fluoroscopy).

Tomographic approaches to NIRF and other imaging can also be used. In general, tomographic methods make use of laser light pulses directed through an animal placed in a homogeneously scattering environment; scattered and fluorescent light that has passed through the animal is recorded at numerous positions. Sophisticated modeling algorithms are then applied to localize the source of excited light in the medium. This approach, termed FMT (fluorescence mediated tomography) is described in Ntziachristos et al., Molecular Imaging, 1(2):82-88, 2002 and Ntziachristos et al., Nature Medicine, 8:757-760, 2002.

A particularly useful light-gathering/image-forming component is an endoscope. Endoscopic devices and techniques that have been used for in vivo optical imaging of numerous tissues and organs, including peritoneum (Gahlen et al., J. Photochem. Photobiol., B 52:131-135, 1999), ovarian cancer (Major et al., Gynecol. Oncol., 66:122-132, 1997), colon (Mycek et al., Gastrointest. Endosc., 48:390-394, 1998; Stepp et al., Endoscopy, 30:379-386, 1998), bile ducts (Izuishi et al., Hepatogastroenterology, 46:804-807, 1999), stomach (Abe et al., Endoscopy 32:281-286, 2000), bladder (Kriegmair et al., Urol. Int., 63:27-31, 1999; Riedl et al., J. Endourol., 13:755-759, 1999), and brain (Ward, J. Laser Appl., 10:224-228, 1998) can be employed in the practice of the present invention. Fluorescence endoscopes are also known in the art (Bhunchet et al., Gastrointest. Endosc., 55, 562-571, 2002; Kobayashi et al., Cancer Lett., 165, 155-159, 2001). One of skill in the art would be able to recognize and make any modifications that may be required, e.g., to optimize the emission and detection spectra of the device for use in imaging a particular organ or tissue region.

Other types of light gathering components useful in the invention are catheter-based devices, including fiber optics devices. Such devices are particularly suitable for intravascular imaging. See, e.g., Tearney et al., Science, 276:2037-2039, 1997; Boppart et al., Proc. Natl. Acad. Sci. USA, 94:4256-4261, 1997.

Still other imaging technologies, including phased array technology (Boas et al., Proc. Natl. Acad. Sci. USA, 91:4887-4891, 1994; Chance, Ann. NY Acad. Sci., 838:29-45, 1998), diffuse optical tomography (Cheng et al., Optics Express, 3:118-123, 1998; Siegel et al., Optics Express, 4:287-298, 1999), intravital microscopy (Dellian et al., Br. J. Cancer, 82:1513-1518, 2000; Monsky et al, Cancer Res., 59:4129-4135, 1999; Fukumura et al., Cell, 94:715-725, 1998), and confocal imaging (Korlach et al., Proc. Natl. Acad. Sci. USA, 96:8461-8466, 1999; Rajadhyaksha et al., J. Invest. Dermatol., 104:946-952, 1995; Gonzalez et al., J. Med., 30:337-356, 1999) can be employed in the practice of the present methods.

Any suitable light-detection/image-recording component, e.g., charge-coupled device (CCD) systems or photographic film, can be used in the invention. The choice of light-detection/image-recording component will depend on factors including type of light gathering/image forming component being used. Selecting suitable components, assembling them into a near infrared imaging system, and operating the system is within the ability of a person of ordinary skill in the art.

The invention is further described in the following examples, which are not intended to limit the scope of the invention described in the claims.

### EXAMPLES

### Example 1

### Preparation of Annexin A5

Annexin A5 was purified substantially as described in U.S. Pat. No. 6,323,313. Briefly, the annexin A5-expressing E. coli clone ACL3 (E. coli strain BL21 (DE3), containing plasmid pET12a.PAPI) was grown in 500 ml TB (Terrific Broth) with 50 μg/ml Kanamycin at 37° C. and 200 rpm overnight. After centrifugation for 10 min at 2500×g at 4° C. the cells were washed with 500 ml of a solution containing 20 mM triethanolamine pH 7.2 and 150 mM NaCl, and then spun down under same conditions. The cells were then resuspended in 500 ml of a solution containing 20 mM triethanolamine and 10 mM CaCl₂. Ten portions of the resulting suspension were sonicated in 50 ml tubes, each for six, 1-minute treatments using a Fisher Scientific Dismembranator 60® set at 8 W, on ice, followed by 20 minutes centrifugation at 22,500×g at 4° C. The precipitate was resuspended in 60 ml of a solution containing 20 mM triethanolamine pH 7.2 and 20 mM EDTA on ice, and then centrifuged for 20 minutes at 22500×g at 4° C. The supernatant was dialyzed (membrane molecular weight cutoff: 12-14 kDa) against 20 mM triethanolamine pH 8.0 at 4° C. overnight, and then filtered through a 0.45 μm filter. The filtrate was applied to a HiTrap Q column (Pharmacia, Piscataway, N.J.), and annexin A5 was eluted with a 100 ml gradient from 0 to 1 M NaCl containing 20 mM triethanolamine pH 8.0 (elution at approximately 0.22 M NaCl). Fractions were pooled based on sodium dodecyl sulfate-polyacrylamide gel electrophoresis (SDS-PAGE) (Laemmli, 1970), and then concentrated and dialyzed against 0.1 M bicarbonate pH 8.0 with Centriprep 10™ columns (3000×g, 4° C.). Purity, assessed by SDS-gel electrophoresis, was 97%.

### Example 2

### Synthesis of Active Annexin A5 Coupled with the Near-Infrared Indocyanine Dye Cy5.5

A solution of 1 mg annexin A5 at 3 mg/ml was prepared and dialyzed against 0.1 M bicarbonate pH 8.0 using Centriprep™ 10 columns (Millipore, Milford Mass.) at 3000 g, 4° C. To synthesize a Cy5.5 labeled annexin V, 333 μl annexin V (3.0 mg/ml dialyzed against 0.1 M bicarbonate pH 8.0) was added to a vial of Cy5.5 N-hydroxysuccinimide ester (Amersham-Pharmacia, Piscataway N.J.). After incubation for 20 minutes at room temperature, the mixture was transferred into a second Cy5.5 vial and incubated for another 40 minutes at room temperature. The conjugate of annexin A5 and Cy5.5 (Cy-annexin) was separated by double spin column separation on BioGel P6 (Bio-Rad, Hercules Calif.) equilibrated with PBS pH 7.4. First, the column was centrifuged at 1000 g for 2 minutes, then annexin-Cy5.5 was added to the column, then the column was spun again at 1000 g for 5 minutes. The eluate was collected and the purification was repeated on another column filled with BioGel P6. The concentration of Cy5.5 dye was determined spectrophotometrically at 675 nm (E678=250,000 M⁻¹cm⁻¹), and protein concentration was determined using the BCA Assay (Pierce-Endogen, Rockford Ill.) and the Cy5.5/annexin molar ratio was calculated. Active Cy5.5-annexin had, on average, 1.1 Cy5.5 molecules bound per mole of annexin.

### Example 3

### Synthesis of Inactive Annexin A5 Coupled with the Near-Infrared Indocyanine Dye Cy5.5

To synthesize annexin-Cy5.5 conjugates with a range of dye to protein ratios, Cy5.5 was solubilized with 7 μl DMSO and added in 1.0, 2.0, or 4.0 μl aliquots to 30 μg annexin A5 (0.1 M Na-carbonate buffer pH 8.0) to give a final volume of 20 Ill. The reaction tubes were incubated for 1.5 hours at room temperature. After adding 30 μl PBS, pH 7.4, the protein was separated from unreacted dye by two successive spin separation using 1 ml Biospin P6 columns equilibrated with PBS pH 7.4 (Bio-Rad).

The conjugate with high Cy5.5 content (2.4 moles Cy5.5 per mol protein, “inactive annexin”) had no binding affinity to apoptotic cells and therefore comprises an excellent control to account for differences in the bioavailability of tumor cells. The molecular weights of annexin A5 and Cy5.5 are 36 and 0.9 kDa, respectively, so active Cy-annexin [total mass=1.1 (0.900)+36 kDa] and inactive Cy-annexin [total mass=2.4(0.90)+36 kDa] differ only by 1.35 kDa.

Samples of active and inactive annexin were treated with trypsin (100 μg/ml, 2 hours at 37° C.) at approximately equal concentration of Cy5.5 dye (500 μM), and fluorescence intensity of Cy5.5 was measured at λex 675 nm/λem 694 nm before and after the treatment with trypsin. The treatment resulted in an increase of Cy5.5 fluorescence from 2500 AU to 12500 AU in both cases, indicating that binding of the dye to the protein resulted in the same degree of fluorescence quenching regardless of the degree of protein modification.

### Example 4

### Synthesis of Active Annexin A5 Coupled with the Near-Infrared Indocyanine Dye Cy7

To synthesize a Cy7 labeled annexin A5, 5 or 10 μl (200 μg Cy7 in 400 μl DMSQ) of Cy7 N-hydroxysuccinimide ester (Amersham-Pharmacia, Piscataway N.J.) were added to 333 μl annexin A5 (3.0 mg/ml dialyzed against 0.1 M bicarbonate pH 8.0). The reaction was incubated for 90 minutes at room temperature. Protein was separated from the unreacted dye by two successive spin separations using 10 mL BioGel P6 columns in PBS pH 7.4 (Bio-Rad, Hercules Calif.). The Cy7 dye concentration was determined spectrophotometrically (E₇₄₇=200000 M⁻¹cm⁻¹). Protein was determined by the BCA method (Bio-Rad, Richmond, Calif.). The conjugates had 0.8 and 1.6 Cy7s attached per mole of annexin A5. Both retained their binding activity.

### Example 5

### Synthesis of Inactive Annexin A5 Coupled with the Near-Infrared Indocyanine Dye Cy7

To synthesize inactive Cy7-labeled annexin A5, 333 μl annexin A5 (3.0 mg/ml dialyzed against 0.1 M sodium bicarbonate pH 8.0) was added to one vial of the Cy7 (1 mg) N-hydroxysuccinimde ester (Amersham-Pharmacia, Piscataway N.J.). The reaction mixture was incubated for 90 minutes at room temperature. The protein was separated from unreacted dye by two successive spin separations using 10 ml BioGel P6 columns in PBS pH 7,4 (Bio-Rad, Hercules Calif.). Cy7 dye concentration was determined spectrophotometrically (E₇₄₇=200000 M⁻¹cm⁻¹). Protein concentration was determined using the BCA method (Bio-Rad, Richmond, Calif.). Inactive conjugate had 4.9 Cy7s attached per mole of annexin A5. The compound has no affinity for membranous phosphatidylserines.

### Example 6

### Synthesis of Inactive Annexin A5 Coupled with the Near-Infrared Dye IR38

A solution of 1 mg annexin A5 at 3 mg/ml was prepared and dialyzed against 0.1 M sodium carbonate pH 8.7 using Centriprep™ 10 columns (Millipore, Milford Mass.) at 3000 g, 4° C. Two mg of IR38 isothiocyanate (LI-COR, Lincoln Nebr.) was dissolved in 40 μl of DMSO, added to the annexin A5 solution, mixed, and incubated for 1 hour at room temperature. The conjugate of annexin A5 and IR38 (annexin-IR38) was separated by double spin column separation on BioGel P6 (Bio-Rad, Hercules Calif.) equilibrated with PBS pH 7.4. First, the column was centrifuged at 1000 g for 2 minutes, the annexin-IR38 was added on the top of the gel in the column, then the column was spun again at 1000 g for 5 minutes. The eluate was collected and the purification was repeated on another column filled with BioGel P6. The concentration of IR38 dye was determined spectrophotometrically at 778 nm (E₇₇₈=179,000 M⁻¹cm⁻¹). The protein concentration was determined using the BCA Assay (Pierce-Endogen, Rockford Ill.) and IR38/annexin molar ratio was calculated. Active annexin-IR38 should have, on average, 1.0-1.5 IR38 molecules bound per mole of annexin.

### Example 7

### Synthesis of C2 Domain of Synaptotagmin I Coupled with the Near-Infrared Indocyanine Dye Cy5.5

First, recombinant C2 domain of synaptotagmin I (11 kDa, C2) is purified using standard methods. To synthesize a Cy5.5 labeled C2, 300 μl C2 (1.0 mg/ml dialyzed against 0.1 M sodium bicarbonate pH 8.0) is added to a vial of the Cy5.5 N-hydroxysuccinimide ester (Amersham-Pharmacia, Piscataway N.J.). After 20 minutes at room temperature the mixture is transferred into a second Cy5.5 vial and incubated for another 40 minutes at room temperature. The protein is separated from unreacted dye by two successive spin separations using 10 ml BioGel P6 columns in PBS pH 7,4 (Bio-Rad, Hercules Calif.). The Cy7 dye concentration is determined spectrophotometrically (E₆₇₈=250000 M⁻¹cm⁻¹), and protein concentration is determined using the BCA method (Bio-Rad, Richmond, Calif.).

### Example 8

### Synthesis of Cy5.5 Labeled Polyclonal Anti-Phosphatidylserine Antibody

To synthesize a Cy5.5 labeled phosphatidylserine antibody, 500 μl phosphatidylserine antibody (1 mg/ml in 0.1 M sodium bicarbonate pH 8.0) is added to a vial of the Cy5.5 N-hydroxysuccinimide ester (Amersham-Pharmacia, Piscataway N.J.). The reaction mixture is incubated for 90 minutes at room temperature, then the protein is separated from the unreacted dye by two successive spin separations using 1 mL BioGel P6 columns in PBS pH 7,4 (Bio-Rad, Hercules Calif.). The protein/Cy ratio is determined as described in Example 6.

### Example 9

### Biological Affinity

To test the biological affinity of annexin A5-Cy5.5 for apoptotic cells, apoptosis was induced in Jurkat T cell lymphoma cells (Clone E6-1, ATCC #TIB-152) by treatment with camptothecin. The Jurkat T cells were grown in RPMI 1640 medium (Vitacell #30-2001) with additional fetal bovine serum (FBS, Vitacell #30-2021)(final concentration 10%). The medium was exchanged every 2 or 3 days. Apoptosis was induced by treatment of cells with 7 μl camptothecin (1 mM in DMSO) per ml culture medium for 5 to 6 hours. Cells were analyzed with a FACS-Calibur® cytometer (Becton Dickinson) after washing and staining with propidium iodide and Annexin A5-FITC (ApoAlert Annexin A5-FITC Apoptosis Kit, Clontech) using a Ca²⁺-containing binding buffer (BB, 1.8 mM CaCl₂, 10 mM HEPES, 150 mM NaCl, 5 mM KCl, pH 7.4).

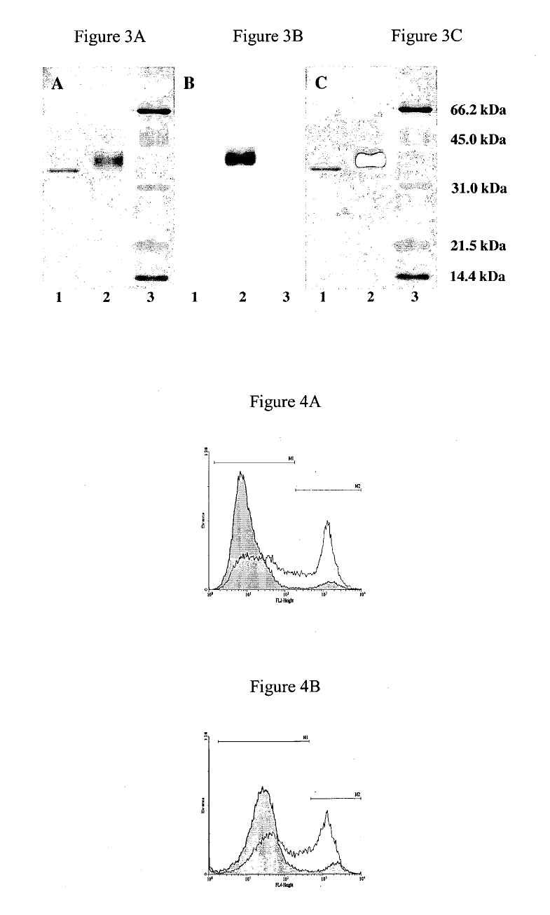

To analyze the quality of synthesis and purification of the annexin A5-Cy5.5, SDS-PAGE protein gel electrophoresis was carried out (FIGS. 3A-3C). FIG. 3A shows the protein silver staining of the gel with unlabeled annexin A5 in lane 1 (running at about 36 kDa), annexin A5-Cy5.5 in lane 2, and a protein size marker in lane 3. The proteins appeared to be highly purified, and the annexin A5-Cy5.5 ran slightly slower compared to unlabeled annexin A5. The near infrared fluorescence image of the same gel in FIG. 3B showed a single band corresponding to the annexin A5-Cy5.5. In FIG. 3C, FIG. 3A and the negative of FIG. 3B are merged.

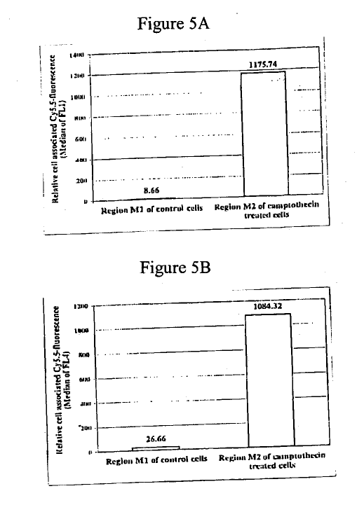

The ability of annexin A5-Cy5.5 to distinguish between apoptotic and non-apoptotic cells was evaluated by FACS analysis of camptothecin-treated Jurkat T cell lymphoma cells, a common model of apoptosis. FIG. 4A shows a FACS histogram in the FACS-Calibur instrument's fluorescence channel 1 (FL1) of untreated (gray area) and camptothecin-treated T cells (white area), stained with annexin A5-FITC. The M1 region, with a low annexin-FITC fluorescence, contained 93% of the untreated, healthy control cells, whereas 43% of the camptothecin-treated cells had a high annexin A5-FITC fluorescence, and therefore represent the apoptotic fraction. FIG. 4B shows the analogous experiment for annexin-Cy5.5 measured in the instrument's NIR fluorescence channel 4 (FL4). Annexin-Cy5.5 also allows for a very clear distinction between healthy and apoptotic cells, but in the near infrared range. The signal difference between non-apoptotic and apoptotic cells was evaluated by the quotient of the medians of the M1 region of non-treated and the M2 region of treated cells (FIGS. 4A and 4B). As indicated by both histograms, the apoptotic cells had significantly higher annexin A5 signals for both labels. The results for annexin A5-FITC and annexin-Cy5.5 are shown in FIGS. 5A and 5B, where the medians of the Ml region of controls and the M2 region camptothecin-treated Jurkat T cells (FIG. 4) are compared. For a non-activatable conjugate, annexin A5-Cy5.5 (FIG. 5B) has a very high signal-to-noise ratio (i.e., 41; apoptotic to non-apoptotic cells), which is, however, lower compared with that of annexin A5-FITC (FIG. 5A) (i.e., 136).

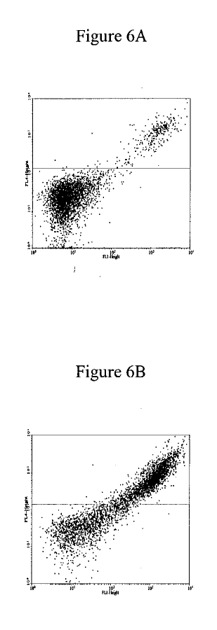

In another FACS experiment, untreated and camptothecin-treated cells were double labeled with annexin A5-FITC and annexin A5-Cy5.5. The resulting dot plots from fluorescence channels 1 and 4 are shown in FIGS. 6A and 6B. 58.9% of the camptothecin-treated cells were in the upper right quadrant, containing the annexin A5 positive, apoptotic cells (6B; 36% were in the lower left quadrant). The lower left quadrant, corresponding to healthy cells, contained 92% of the non-treated control cells, while the upper right quadrant contained only 7.4% of the non-treated cells (6A).

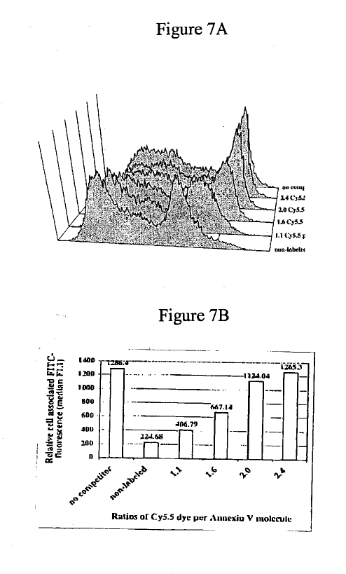

The impact of coupling the Cy5.5 dye for the binding activity of annexin A5 was evaluated in a FACS competition assay. Camptothecin treated T cells were pre-incubated for 10 minutes with a 10-fold access of different preparations of annexin A5-Cy5.5 that had either no Cy5.5 or increasing ratios of Cy5.5 dye per annexin molecule (ranging from 1.1 to 2.4). After adding annexin A5-FITC and incubation for another 10 minutes, a FACS analysis in the FITC channel (FACS channel FL1) was performed (histogram in FIG. 7A). Competition, and therefore the binding affinity, was found to decrease dramatically with increasing amounts of Cy5.5 coupled to annexin A5. The highest ratio, Cy5.5_{2.4}-annexin A5 (2.4 moles dye/mole protein) gave a very low displacement of 0.98 compared to a reference value of 1.0 for no added annexin A5 (the fluorescence height is almost as high as in the sample without a competitor) (chart in FIG. 7B). The median of relative fluorescence of the annexin A5-Cy5.5 with 2.4 Cy5.5 dyes per protein (i.e., 1265) is very similar to that of the sample with no competitor (i.e., 1286), which indicates that the binding affinity is very low. Thus, Cy5.5-24-annexin A5 is referred to as “inactive Cy5.5-annexin A5”. In comparison, Cy5.5_{1.1}-annexin A5 gave a displacement of 0.32 compared to a reference value of 0.17 for unlabeled annexin A5 and is referred to as “active Cy-annexin A5” herein. Inactive Cy-annexin was used as a control, to demonstrate that the NIRF obtained in vivo with active Cy-annexin reflects PS binding rather than non-specific accumulation.

### Example 10

### Stable Transfection of Cells with DsRed2

cDNA encoding DsRed2 was obtained by excising the DNA insert from the pDsRed2-1 vector (Clontech, Palo Alto, Calif.) using Hind III and Not I endonucleases and cloned into the eukaryotic expression vector pcDNA3 (Invitrogen, Carlsbad, Calif.). Cells at 50-60% confluence were transfected using Maxfect™ (Molecula Research, Herndon, Va., USA) at the ratio of 1 g DNA:3 μg Maxfect™ reagent. Twenty-four or 72 hours after transfection, cells were trypsinized and sorted using FACSVantage™ (Beckton-Dickinson).

DsRed2-transfected lines were maintained in 10% FCS, DMEM supplemented with 1 mg/ml G418 (Invitrogen-Gibco BRL, Grand Island, N.Y.).

### Example 11

### Screening of Apoptosis in Tumors Using Near-Infrared Fluorescent Imaging in Animal Models in Vivo: General Methods

GFP- or DsRed2-expressing tumors were propagated in nu/nu mice by injecting 2×10⁵ cells in 25 μl of serum-free cell culture medium in a suitable location subcutaneously in anesthetized animals. Animals were evaluated on the 7-10^{th} day after the inoculation, when the tumors reached 3-4 mm in diameter. Prior to optical imaging, fluorescent-annexin A5 conjugates were injected intravenously via the tail vein at a dose not exceeding 75 μg annexin A5/animal, i.e. less than 3.1 mg annexin A5/kg. Tumor apoptosis was induced by administration of cyclophosphamide (CPA, Mead Johnson, Princeton, N.J.) given as a single intraperitoneal injection at 170 mg/kg. The animals were anesthetized and subjected to NIRF imaging at 24 hours after chemotherapy administration. Optical reflectance NIRF imaging was performed using a light-tight compartment equipped with a halogen lamp or other suitable NIR excitation source and an excitation filter set suitable for GFP, DsRed2 as well as Cy 5.5, Cy7 and IR38 (Omega Optical, Brattleboro, Vt.)(see FIG. 1). Excitation light was distributed over the field of view (FOV) using light diffusers. The anesthetized animals were positioned on the glass platen using a template enabling reproducible imaging of animals at a fixed distance from the excitation source. Animals were imaged with tumors facing the glass platen surface. Fluorescent images were collected using a CCD (Kodak, Rochester, N.Y. or similar) equipped with a f/1.2 12.5-75 mm zoom lens and emission filters (Omega Optical). Optical images were acquired in anesthetized animals in the following sequence: 1) visible light image (to outline the animal), 2) fluorescent image of the tumor marker (GFP or DsRed2), 3) fluorescent image in the NIRF channel before NIRF-annexin conjugate injection, to obtain background reflectance image, and 4) fluorescent image in the NIRF channel at various times after the NIRF-annexin conjugate injection. Images were acquired as TIFF files and processed using commercially available software. Fluorescence signal changes were determined by subtracting background (pre-injection) signal.

### Example 12

### Detection of Tumor Cell Apoptosis after in Vivo Treatment with Cyclophosphamide in an Animal Model of Cancer Using Near-Infrared (NIRF) Imaging: Gliosarcoma

9L-GFP gliosarcoma tumor line constitutively expressing GFP was propagated in DMEM/10% FCS. Subcutaneous tumors were implanted in nu/nu mice (25-28 g; Jackson Laboratories, Bar Harbor, Me.) by inoculating 5×10⁵ cells/0.025 ml into the right ear pinna. Tumor volumes were determined by caliper measurements of three orthogonal diameters (X, Y, Z), using the formula V=p(X×Y×Z)/6. GFP expression was used as an independent optical marker for tumor localization.

All mice bearing 9L-GFP (n=4) were divided into 2 groups: a) treated with a single IP injection of 170 mg CPA/kg i.p.) (n=2); and b) control, saline treated (n=2). Cyclophosphamide (MeadJohnson) was diluted with sterile saline solution to 17 mg/ml just before dosing.

On day 0, animals were injected with CPA or saline as described above. On day 1, all tumor-bearing animals were anaesthetized, and the optical imaging was performed using visible light, GFP, and NIRF channels prior to fluorescent conjugate administration. Imaging was performed using the modified chemiluminescent imaging system (Eastman Kodak, Rochester, N.Y.). After the first imaging, active annexin A5-Cy5.5 conjugate was injected in the tail veins of the animals. Post NIRF-conjugate imaging was performed immediately after the injection and at 1.5, 3, and 24 hour time points. Animals were anaesthetized before each imaging session.

Images were analyzed using IP Lab Spectrum software (Scanalytics, Inc., Fairfax, Va.). The regions of interest (ROI) were chosen using GFP imaging data to localize tumor margins and NIRF signal intensity was measured in the tumors.

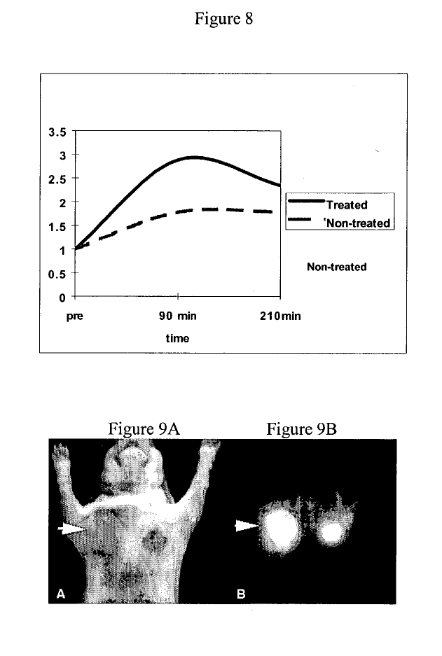

In both CPA-treated and control groups a time-dependent increase of fluorescence intensity measured in NIRF channel was evident during the first 200 minutes after the injection of active Cy annexin. However, in CPA-treated animals the drug metabolite induces elevated cell death rate in tumors and this effect was confirmed by measuring fluorescence intensity values after annexin injection (FIG. 8). An overall 30-40% higher fluorescence intensity of CPA-treated tumors vs. control tumors was measured. In a separate experiment, using a bilateral Lewis lung carcinoma model, we observed a strong visual enhancement of tumor signal intensity after CPA treatment (FIGS. 9A and 9B).

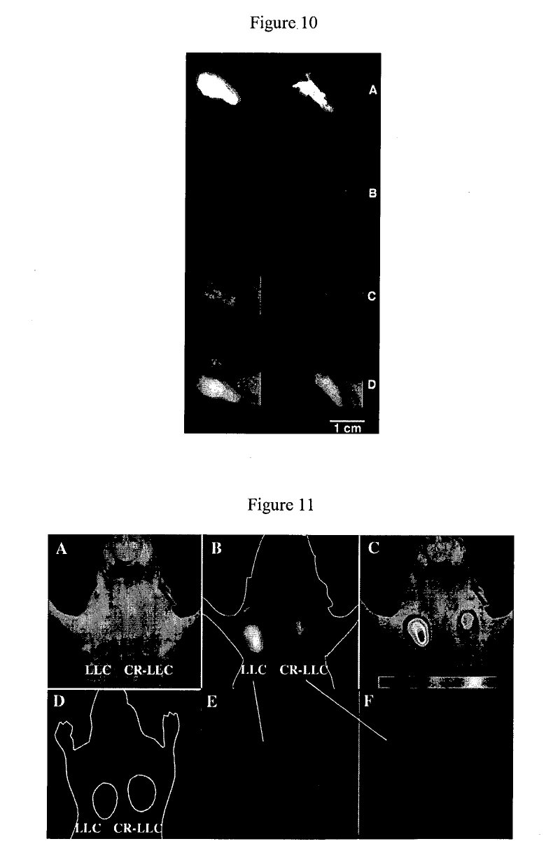

FIG. 10 depicts two animals from independent experiments (the right and left columns). Green fluorescent protein signal provided an outline of tumor margins (Row A). NIRF prior to injection of the active annexin 5A is shown in Row B. Consistent with results obtained with Lewis lung carcinoma, cyclophosphamide treatment (Row D) produced a higher tumoral NIRF than obtained with animals before the treatment (Row C). Histology of 9L-GFP suggested that a substantial number of apoptotic cells in these tumors are endothelial cells of tumor blood vessels. This was confirmed by co-staining of annexin A5-positive cells with fluorescent anti-CD31 (anti-PECAM-1) antibodies.

### Example 13

### Detection of Tumor Cell Apoptosis after in vivo Treatment with Cyclophosphamide in an Animal Model of Cancer Using Near-Infrared (NIRF) Imaging: Bilateral Lewis Lung Carcinoma Model

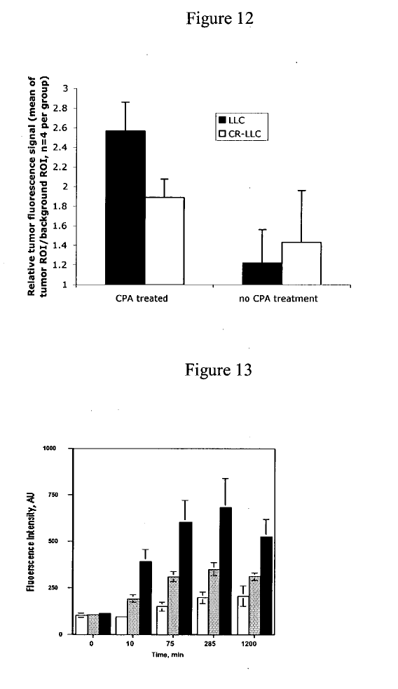

The active Cy annexin and inactive Cy annexin were tested in vivo in a bilateral tumor model. Nude mice implanted with a cyclophosphamide sensitive Lewis lung carcinoma (LLC) and cyclophosphamide (CPA) resistant tumor (CR-LLC) were injected with NIRF-labeled Annexin A5 and imaged using a surface reflectance method as described herein. As is shown in FIGS. 11B and 11C, the chemosensitive LLC tumor had a far higher fluorescence than the chemoresistant CR-LLC tumor, and tumor NIRF obtained with the active-Cy annexin reflected PS binding, since inactive-Cy annexin gave no signal (compare FIGS. 11B and 11D). Further experiments with additional animals were performed to corroborate the striking visual impression of FIGS. 11A-F. FIG. 12 shows the results obtained in four additional animals bearing LLC and CR-LLC tumors. After treatment with CPA, the chemosensitive LLC had a specific signal of 2.56±0.29 compared to 1.89±0.19 for CR-LLC, which were significantly different (p=0.002). Hence, after CPA treatment the tumor signal was a function of tumor chemosensitivity.

CPA treatment significantly increased the tumor signal of both the LLC and CR-LLC tumors, and the statistical significance of this increase was higher for the LLC than the CR-LLC tumor. CPA treatment increased the tumor fluorescence of the chemosensitive LLC tumor (1.22±0.34 to 2.56±0.29, p=0.001, unpaired Student's t-test), while the chemoresistant CR-LLC tumors had a more modest signal increase (1.43±0.53 to 1.89±0.19, (p=0.183, unpaired Student's t-test). When inactive Cy-annexin was injected into LLC and CR-LLC tumor bearing animals, with or without CPA treatment, tumor NIRF/background NIRF values ranged from 0.99 to 1.17 and the magnitude of non-tumor signal intensity (background) was similar using active Cy-annexin or inactive Cy-annexin (FIG. 11D).

The time dependence of tumor NIRF signal intensity after cyclophosphamide treatment was examined in a chemosensitive Lewis lung carcinoma transfected to express DsRed2 marker protein (FIG. 13; open bars: cyclophosphamide (CPA) treated/inactive Cy-annexin (n=3); dotted bars: no CPA/active Cy-annexin V (n=6); black bars: CPA treated/active Cy-annexin (n=6)). The animals were treated with a single dose of cyclophosphamide, followed by injection of active Cy-annexin or inactive Cy-annexin (as in FIGS. 11A-F and 12). Signal intensity was measured from a region-of-interest defined by DsRed2 fluorescence and is expressed as the median signal with the background fluorescence subtracted. Results are presented as mean±SD. Signal intensity was measured at various times after injection of the Cy-annexins. Tumor NIRF increased with time after the injection of active Cy-annexin A5, reaching a plateau at 75-285 minutes post injection. As in FIG. 12, cyclophosphamide treatment increased tumor NIRF and inactive Cy-annexin served as control for non-specific accumulation of annexin in the tumor.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A non-invasive method of detecting a site of apoptosis in vivo in a subject, the method comprising:
administering a composition to the subject comprising a fluorochrome conjugated to a moiety that binds specifically to apoptotic cells, and
obtaining a fluorescence image of at least part of the subject to detect the site of apoptosis in the subject.

2. The method of claim 1, wherein the moiety is a protein or an active fragment thereof.

3. The method of claim 2, wherein the protein is selected from the group consisting of annexin and synaptotagmin.

4. The method of claim 2, wherein the active fragment comprises the C2 domain of synaptotagmin.

5. The method of claim 1, wherein the moiety is an anti-aminophospholipid antibody or an active fragment thereof.

6. The method of claim 5, wherein the antibody is an anti-phosphatidylserine antibody or an active fragment thereof.

7. The method of claim 5, wherein the antibody is an anti-phosphatidylethanolamine antibody or an active fragment thereof.

8. The method of claim 1, wherein the fluorochrome fluoresces in the near-infrared region.

9. The method of claim 1, wherein the fluorochrome is selected from the group consisting of Cy5, Cy5.5, Cy7, ALEXA FLUOR 680, ALEXA FLUOR 700, ALEXA FLUOR 750, IRDye38, IRDye78, IRDye80, indocyanine green, LaJolla Blue, and Licor NIR.

10. The method of claim 1, wherein the subject is an animal.

11. The method of claim 1, wherein the subject is a mammal.

12. The method of claim 11, wherein the mammal is a mouse.

13. The method of claim 1, wherein the subject is a human.

14. The method of claim 1, wherein the administering is orally, parenterally, by inhalation, topically, rectally, nasally, buccally, vaginally, via an implanted reservoir, via a catheter, or through a needle to a tissue.

15. The method of claim 1, wherein the obtaining is by near-infrared fluorescence (NIRF) imaging.

16. The method of claim 1, wherein the NIRF imaging is fluorescence mediated tomography (FMT).

17. The method of claim 1, wherein the NIRF imaging is surface reflectance imaging.

18. The method of claim 1, wherein the NIRF imaging uses an endoscope.

19. A conjugate comprising:
a moiety that binds specifically to apoptotic cells, and
a fluorochrome covalently bound to the moiety,
wherein the moiety and the fluorochrome are present in a stoichiometry of at least 1:2.

20. The conjugate of claim 19, wherein the moiety is annexin or an active fragment thereof or synaptotagmin or an active fragment thereof.

21. The conjugate of claim 19, wherein the moiety is an anti-aminophospholipid antibody or active fragment thereof.

22. The conjugate of claim 19, wherein the fluorochrome emits near-infrared fluorescence.

23. The conjugate of claim 22, wherein the fluorochrome is selected from the group consisting of Cy5, Cy5.5, Cy7, ALEXA FLUOR 680, ALEXA FLUOR 700, ALEXA FLUOR 750, IRDye38, IRDye78, IRDye80, indocyanine green, LaJolla Blue, and Licor NIR.

24. A conjugate comprising annexin and a fluorochrome selected from the group consisting of ALEXA FLUOR 680, ALEXA FLUOR 700, ALEXA FLUOR 750, IRDye38, IRDye78, IRDye80, indocyanine green, LaJolla Blue, and Licor NIR.

25. A conjugate comprising synaptotagmin or an anti-aminophospholipid antibody and a fluorochrome selected from the group consisting of Cy5, Cy5.5, Cy7, ALEXA FLUOR 680, ALEXA FLUOR 700, ALEXA FLUOR 750, IRDye38, IRDye78, IRDye80, indocyanine green, LaJolla Blue, and Licor NIR.

26. The conjugate of claim 25, wherein the anti-aminophospholipid antibody is an anti-phosphatidylserine antibody.

27. The conjugate of claim 25, wherein the anti-aminophospholipid antibody is an anti-phosphatidylethanolamine antibody.
